(19) United States
(12) Patent Application Publication
(10) Pub. No.: US 2012/0103336 A1
(43) Pub. Date: May 3, 2012

(54) Ventilator System and Method

(75) Inventor: Gerard Evers, Saint Priest (FR)

(73) Assignee: GENERAL ELECTRIC COMPANY, Schenectady, NY (US)

(21) Appl. No.: 12/915,287

(22) Filed: Oct. 29, 2010

(51) Int. Cl. A61M 16/00

(52) U.S. Cl. 128/204.21

(57) ABSTRACT

A ventilator is disclosed herein. The ventilator may include a blower, and a controller operatively connected to the blower. The controller is configured to automatically identify an optimal target inspiratory and expiratory pressure level for the treatment of a sleep related breathing disorder. The controller is also configured to regulate the operation of the blower in a manner adapted to deliver the optimal target inspiratory and expiratory pressure level.

### FIELD OF THE INVENTION

This disclosure relates generally to the measurement and control of breathing gas administration into humans, and more specifically automatic, adaptive control mechanisms for detection and treatment of breathing disorders.

### BACKGROUND OF THE INVENTION

Respiratory failure includes all forms of insufficient ventilation with respect to metabolic need whether occurring during wake or periods of sleep. The condition is highly disabling in terms of reduced physical capacity, cognitive dysfunction in severe cases and poor quality of life. Patients with respiratory failure therefore experience significant daytime symptoms but in addition, the majority of these cases experience a general worsening of their condition during state changes such as sleep

Medical ventilators systems may be implemented to treat respiratory failure like obstructive or resistive airway diseases, or specific sleep related breathing disorders such as sleep apnea. The primary function of the medical ventilator system is to maintain suitable pressure and flow of gases inspired and/or expired by the patient. A category of ventilator designated hereafter as a Bi-level positive pressure ventilator provides two potentially distinct pressure levels, Inspiratory Positive Airway Pressure (IPAP) and Expiratory Positive Airway Pressure (EPAP). IPAP is administered during the inhalation phase while EPAP is given during the exhalation phase.

One problem with conventional Bi-level positive pressure ventilator systems relates to the difficulty associated with identifying suitable inspiratory and expiratory pressure levels. If the inspiratory and expiratory pressure levels established by the ventilator system are either too high or too low, the resultant treatment may be ineffective. This problem is complicated by the fact that a suitable pressure level may differ based on the time of day or night, and may also change over time. Clinically applicable IPAP and EPAP levels typically need to be identified, which may be done during a sleep study conducted in a sleep laboratory. This nighttime identification of pressure settings requires the presence of clinical staff at a time when they might not be available.

### BRIEF DESCRIPTION OF THE INVENTION

The above-mentioned shortcomings, disadvantages and problems are addressed herein which will be understood by reading and understanding the following specification.

In an embodiment, a ventilator includes a blower, and a controller operatively connected to the blower. The controller is configured to automatically identify an optimal target inspiratory and expiratory pressure level for the treatment of a sleep related breathing disorder. The controller is also configured to regulate the operation of the blower in a manner adapted to synchronously deliver the optimal target inspiratory and expiratory pressure level.

In another embodiment, a ventilator system includes a breathing circuit, and a Bi-level positive pressure ventilator pneumatically coupled with the breathing circuit. The Bi-level positive pressure ventilator includes a blower, and a controller operatively connected to the blower. The controller is configured to automatically identify an optimal target inspiratory and expiratory pressure level based on an approximation of the respective pressure levels minimally sufficient for maintaining the patient's airway in an asymptomatic state. The controller is also configured to regulate the operation of the blower in a manner adapted to synchronously deliver the optimal target inspiratory and expiratory pressure levels.

In another embodiment, a method for automatically identifying and providing an optimal inspiratory and expiratory pressure level for the treatment of sleep related breathing disorders includes providing a ventilator comprising a blower and a controller. The method also includes implementing the ventilator controller to approximate the inspiratory and expiratory pressure levels minimally sufficient for maintaining a patient's airway in an asymptomatic state. The method also includes implementing the ventilator controller to automatically identify optimal target inspiratory and expiratory pressure levels based on the approximation of inspiratory and expiratory pressure levels minimally sufficient for maintaining a patient's airway in an asymptomatic state. The method also includes implementing the ventilator controller to regulate the operation of the blower in a manner adapted to synchronously deliver the optimal target inspiratory and expiratory pressure levels.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a ventilator system in accordance with an embodiment; and

FIG. 2 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

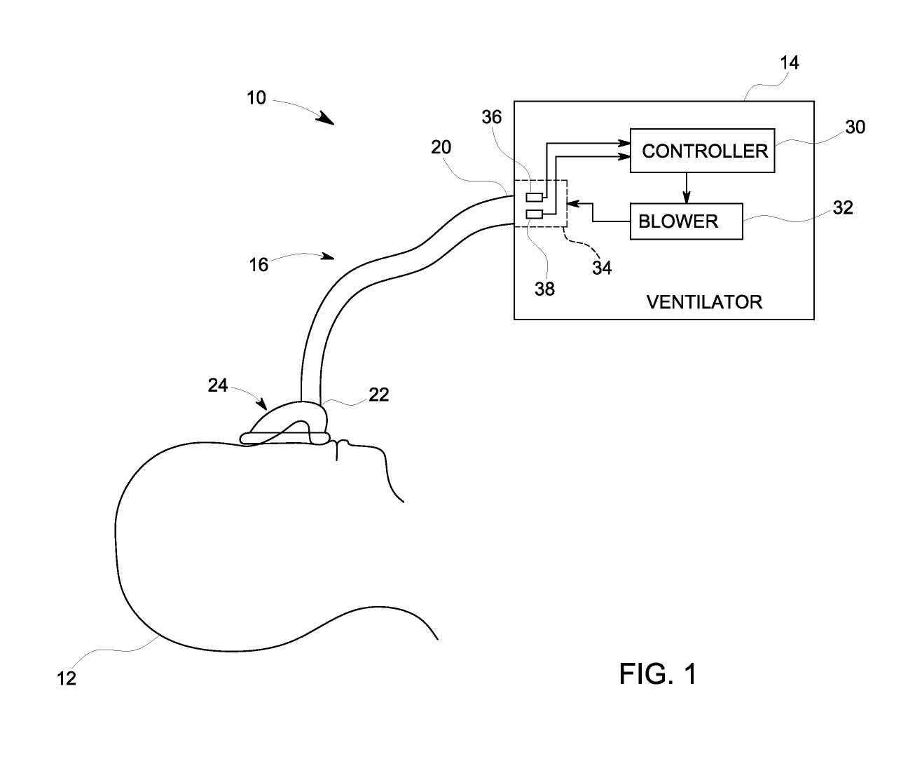

Referring to FIG. 1, a schematically illustrated ventilator system 10 is shown connected to a patient 12 in accordance with an exemplary embodiment. The ventilator system 10 includes a ventilator 14, and a breathing circuit 16. The ventilator 14 will hereinafter be described in accordance with an embodiment as a portable Bi-level positive pressure device adapted for the in-home treatment of sleep related breathing disorders. It should, however, be appreciated that other types of ventilators may be envisioned.

The breathing circuit 16 is adapted to pneumatically couple the ventilator 14 with the patient 12. The breathing circuit 16 includes a first terminal end 20 adapted for attachment to the ventilator 14, a second terminal end 22, and a patient interface 24. The patient interface 24 is the portion of the breathing circuit 16 that is in direct contact with the patient 12. According to the embodiment depicted and described hereinafter, the patient interface 24 is a nasal mask, however it should be appreciated that other known devices (e.g., oral mask, endotracheal tube, etc.) may also be implemented.

The ventilator 14 provides breathing gasses that are transferred to the patient 12 via the breathing circuit 16. The ventilator 14 includes a controller 30, a blower 32, and a connector 34. The connector 34 is adapted to receive the first terminal end 20 of the breathing circuit 16. The ventilator 14 may optionally include a pressure sensor 36 and a flow sensor 38 disposed at or near the connector 34 such that they remain in pneumatic communication with the breathing circuit 16. The sensors 36, 38 may alternatively be included as part of the breathing circuit 16.

According to an embodiment, the controller 30 is adapted to regulate the operation of the blower 32 based on feedback from the pressure sensor 36 and/or the flow sensor 38. The blower 32 may be operable to transfer a fluid through the breathing circuit 16 to the patient 12 at a selectable rate, and to thereby maintain suitable pressure and flow of gases inspired and expired by the patient 12. For purposes of this disclosure, the term fluid should be defined in a non-limiting manner to include any substance that continually deforms or flows under an applied shear stress such as, for example, a liquid or a gas. The blower 32 may comprise any known device adapted to facilitate the transfer of a fluid such as, for example, a pump or a fan.

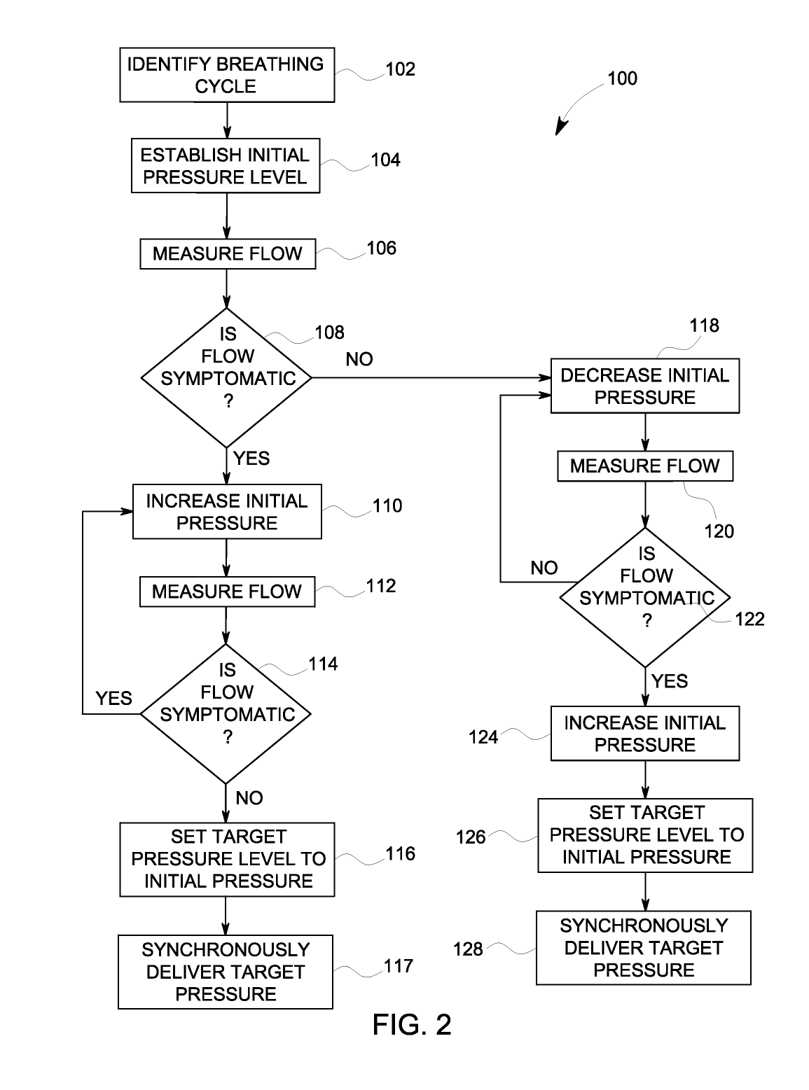

Referring to FIG. 2, a flow chart illustrating an algorithm 100 is shown in accordance with an embodiment. The technical effect of the algorithm 100 is to automatically identify and establish optimal target inspiratory and expiratory pressure levels for the ventilator system 10 (shown in FIG. 1). The optimal target inspiratory and expiratory pressure levels should be defined as those values best suited to the treatment of a specific condition. Referring to the exemplary embodiment in which the ventilator system 10 comprises a Bi-level positive pressure device, optimal target inspiratory and expiratory pressure levels may include pressure levels minimally capable of preventing airway occlusion such that the patient's airway remains open while avoiding discomfort associated with excess pressure. According to one embodiment, the at least a portion of the algorithm 100 comprises a computer program stored on a computer-readable storage medium. The individual blocks 102-128 represent steps that can be performed by the controller 30 (shown in FIG. 1).

Referring now to FIGS. 1 and 2, at step 102 the algorithm 100 is configured to identify a patient breathing cycle (i.e., inspiratory or expiratory). The breathing cycle may, for example, be identified based on feedback from the flow sensor 38 indicating the direction of flow. More precisely, flow in a direction toward the patient 12 is indicative of the inspiratory cycle, and flow in a direction away from the patient 12 is indicative of the expiratory cycle.

At step 104, the algorithm 100 is configured to establish an initial pressure level for the breathing cycle identified at step 102. The initial pressure level may be intentionally low as a starting point or set manually by the clinical staff If, for example, an inspiratory breathing cycle is identified at step 102, the algorithm 100 may establish an initial inspiratory pressure level of 10 cm H2O. Similarly, if an expiratory breathing cycle is identified at step 102, the algorithm 100 may establish an initial expiratory pressure level of 5 cm H2O.

At step 106, the algorithm 100 measures fluid flow through the pneumatic circuit 16. According to an embodiment, the flow sensor 38 may be implemented at step 106 to measure fluid flow.

At step 108, the algorithm 100 determines whether fluid flow through the pneumatic circuit 16 is symptomatic. For purposes of this disclosure, a fluid flow is considered symptomatic when there is zero or limited fluid flow (e.g., an obstructed or restricted airway), reduced peakflow, tachypnea, bradypnea or any sort of irregular breathing in terms of amplitude, frequency or timing. Similarly, a fluid flow is considered asymptomatic in the absence of any of the above-cited conditions. Symptomatic breathing can be measured and calculated in a known manner using methods not limited to pressure, flow, breathing rate, inspiratory or expiratory time, flow acceleration or deceleration or any combination of these values.

It should be appreciated that symptomatic fluid flow during the inspiratory or expiratory breathing cycle is potentially indicative of a respiratory failure. Accordingly, step 108 is intended to assess the status of a patient's airway (e.g., open, restricted or occluded), and to thereby identify the potential need for increased pressure to alleviate the symptomatic breathing. If at step 108 it is determined that the fluid flow through the pneumatic circuit 16 is symptomatic, the algorithm 100 proceeds to step 110. If at step 108 it is determined that the fluid flow through the pneumatic circuit 16 is not symptomatic, the algorithm 100 proceeds to step 118. According to an embodiment, the controller 30 may be implemented to determine whether fluid flow is symptomatic based on measured data from the flow sensor 38.

Steps 110-117 are responsive to a determination at step 108 that the patient's breathing is symptomatic and the initial pressure level may be too low. At step 110, the initial pressure level is increased. The pressure level may be increased in small increments over multiple breaths to minimize patient discomfort. According to an embodiment, the controller 30 increases the speed of the blower 28 in order to increase pressure level.

At step 112, the algorithm 100 measures fluid flow through the pneumatic circuit 16. At step 114, the algorithm 100 determines whether fluid flow through the pneumatic circuit 16 is symptomatic. If at step 114 it is determined that the fluid flow through the pneumatic circuit 16 is symptomatic, the algorithm 100 returns to step 110. If at step 108 it is determined that the fluid flow through the pneumatic circuit 16 is not symptomatic, the algorithm 100 proceeds to step 116.

At step 116, the target pressure level for a given breathing cycle is set to the currently established initial pressure level for that breathing cycle. It should be appreciated that a target pressure level established in the manner described is only minimally capable of alleviating a symptomatic breathing condition. By minimizing the requisite pressure level, the patient 12 can be treated (e.g., for sleep apnea) without sacrificing patient comfort such as with an unnecessarily high delivered pressure. According to an embodiment, the controller 30 may apply and maintain the set target pressure level by regulating the speed of the blower 32 in response to feedback from the pressure sensor 36.

At step 117, the set target pressure level is synchronously delivered to the patient 12. According to an embodiment, at step 117 the controller 30 may be configured to regulate the operation of the blower 32 in a manner adapted to deliver the set target pressure level synchronously with patient's breathing. According to another embodiment, at step 117 the controller 30 may be configured to identify the patient's breathing cycle in the manner described at step 102 in order to ensure the set target pressure level is synchronously delivered. For purposes of this disclosure, a synchronously delivered target pressure refers to the delivery of a target inspiratory pressure level exclusively during the patient's inspiratory phase, and the delivery of a target expiratory pressure level exclusively during the patient's expiratory phase. The target pressure level may be synchronously delivered for all future breathing cycles until an update becomes necessary.

Steps 118-128 are responsive to a determination at step 108 that the patient's breathing is asymptomatic and the initial pressure level may be too high. At step 118, the initial pressure level is decreased. The pressure level may be decreased in small increments over multiple breaths to minimize patient discomfort. According to an embodiment, the controller 30 may decrease the speed of the blower 28 in order to decrease pressure level.

At step 120, the algorithm 100 measures fluid flow through the pneumatic circuit 16. At step 122, the algorithm 100 determines whether fluid flow through the pneumatic circuit 16 is symptomatic. If at step 122 it is determined that the fluid flow through the pneumatic circuit 16 is not symptomatic, the algorithm 100 returns to step 118. If at step 122 it is determined that the fluid flow through the pneumatic circuit 16 is symptomatic, the algorithm 100 proceeds to step 124.

At step 124, the initial pressure level is increased. The pressure level may be increased in small increments over multiple breaths to minimize patient discomfort. According to an embodiment, the controller 30 increases the speed of the blower 28 in order to increase pressure level.

At step 126, the target pressure level for a given breathing cycle is set to the currently established initial pressure level for that breathing cycle. It should be appreciated that a target pressure level established in the manner described is only minimally capable of alleviating a symptomatic breathing condition. By minimizing the requisite pressure level, the patient 12 can be treated for sleep related breathing disorders without sacrificing patient comfort such as with an unnecessarily high delivered pressure level. According to an embodiment, the controller 30 may apply and maintain the set target pressure level by regulating the speed of the blower 32 in response to feedback from the pressure sensor 36.

At step 128, the set target pressure level is synchronously delivered to the patient 12. According to an embodiment, at step 128 the controller 30 may be configured to regulate the operation of the blower 32 in a manner adapted to deliver the set target pressure level synchronously with patient's breathing. According to another embodiment, at step 128 the controller 30 may be configured to identify the patient's breathing cycle in the manner described at step 102 in order to ensure the set target pressure level is synchronously delivered. The target pressure level may be synchronously delivered for all future breathing cycles until an update becomes necessary.

It is envisioned that the algorithm 100 may be initiated when a given patient uses the ventilator system 10 for the first time in order to establish optimal target inspiratory and expiratory pressure levels. The algorithm 100 may be configured to automatically update target pressure levels on a periodic basis (e.g., weekly or monthly) in order to account for physiology changes or changes in the severity of a sleep related breathing disorder. Alternatively, the algorithm 100 may be manually activated by a patient such as with a button (not shown) included on the ventilator system 10.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A ventilator comprising:
a blower; and
a controller operatively connected to the blower, said controller configured to:
automatically identify an optimal target inspiratory pressure level for the treatment of a sleep related breathing disorder;
automatically identify an optimal target expiratory pressure level for the treatment of the sleep related breathing disorder; and
regulate the operation of the blower in a manner adapted to synchronously deliver the optimal target inspiratory and expiratory pressure level.

2. The ventilator of claim 1, wherein the controller is configured to produce an initial inspiratory and expiratory pressure level.

3. The ventilator of claim 2, wherein the controller is configured to assess the status of the patient's airway based on feedback from a flow sensor.

4. The ventilator of claim 3, wherein the controller is configured to incrementally adjust the initial inspiratory and expiratory pressure level until the patient's breathing is asymptomatic.

5. The ventilator of claim 4, wherein the controller is configured to automatically identify an optimal target inspiratory and expiratory pressure level as those respective pressure levels minimally sufficient for maintaining the patient's breathing in an asymptomatic state.

6. The ventilator of claim 1, wherein the controller is configured to regulate the operation of the blower in a manner adapted to deliver the optimal target inspiratory and expiratory pressure level based on feedback from a pressure sensor.

7. The ventilator of claim 1, further comprising a pressure sensor and a flow sensor.

8. A ventilator system comprising:
a breathing circuit; and
a Bi-level positive pressure ventilator pneumatically coupled with the breathing circuit, said Bi-level positive pressure ventilator adapted for the treatment of sleep related breathing disorders, said Bi-level positive pressure ventilator comprising:
a blower; and
a controller operatively connected to the blower, said controller configured to:
automatically identify an optimal target inspiratory and expiratory pressure level based on an approximation of the respective pressure levels minimally sufficient for maintaining the patient's airway in an asymptomatic state; and
regulate the operation of the blower in a manner adapted to synchronously deliver the optimal target inspiratory and expiratory pressure level.

9. The ventilator system of claim 8, wherein the controller is configured to produce an initial inspiratory and expiratory pressure level.

10. The ventilator system of claim 9, wherein the controller is configured to assess the status of the patient's airway based on feedback from a flow sensor.

11. The ventilator system of claim 10, wherein the controller is configured to incrementally adjust the initial inspiratory and expiratory pressure level until the patient's breathing is asymptomatic.

12. The ventilator system of claim 8, wherein the controller is configured to regulate the operation of the blower in a manner adapted to deliver the optimal target inspiratory and expiratory pressure level based on feedback from a pressure sensor.

13. The ventilator system of claim 8, further comprising a pressure sensor and a flow sensor in pneumatic communication with the blower.

14. A method for automatically identifying and providing an optimal inspiratory and expiratory pressure level for the treatment of sleep related breathing disorders comprising:
providing a ventilator comprising a blower and a controller;
implementing the ventilator controller to approximate the inspiratory and expiratory pressure levels minimally sufficient for maintaining a patient's breathing in an asymptomatic state;
implementing the ventilator controller to automatically identify an optimal target inspiratory and expiratory pressure level based on the approximation of inspiratory and expiratory pressure levels minimally sufficient for maintaining a patient's breathing in an asymptomatic state; and
implementing the ventilator controller to regulate the operation of the blower in a manner adapted to synchronously deliver the optimal target inspiratory and expiratory pressure level.

15. The method of claim 14, wherein said implementing the ventilator controller to approximate the inspiratory and expiratory pressure levels minimally sufficient for maintaining a patient's breathing in an asymptomatic state comprises providing an initial inspiratory and expiratory pressure level, and incrementally adjusting the initial inspiratory and expiratory pressure levels based on feedback from a flow sensor.

16. The method of claim 14, wherein said implementing the ventilator controller to regulate the operation of the blower in a manner adapted to provide the optimal target inspiratory and expiratory pressure level comprises implementing the ventilator controller to regulate the operation of the blower in a manner adapted to provide the optimal target inspiratory and expiratory pressure level based on feedback from a pressure sensor.
